(19) United States
(12) Patent Application Publication
(10) Pub. No.: US 2013/0023765 A1
(43) Pub. Date: Jan. 24, 2013

(54) APPARATUS AND METHOD FOR QUANTITATIVE NONCONTACT IN VIVO FLUORESCENCE TOMOGRAPHY USING A PRIORI INFORMATION

(71) Applicants: Orhan Nalcioglu, Newport Coast, CA (US); Gultekin Gulsen, Irvine, CA (US); Yuting Lin, Mission Viejo, CA (US)

(72) Inventors: Orhan Nalcioglu, Newport Coast, CA (US); Gultekin Gulsen, Irvine, CA (US); Yuting Lin, Mission Viejo, CA (US)

(73) Assignee: THE REGENTS OF THE UNIVERSITY OF CALIFORNIA, Oakland, CA (US)

(21) Appl. No.: 13/340,323

(22) Filed: Dec. 29, 2011

Related U.S. Application Data
(60) Provisional application No. 61/430,036, filed on Jan. 5, 2011.

(51) Int. Cl. A61B 6/02

(52) U.S. Cl. 600/427

(57) ABSTRACT

An apparatus for providing an integrated tri-modality system includes a fluorescence tomography subsystem (FT), a diffuse optical tomography subsystem (DOT), and an x-ray tomography subsystem (XCT), where each subsystem is combined in the integrated tri-modality system to perform quantitative fluorescence tomography with the fluorescence tomography subsystem (FT) using multimodality imaging with the x-ray tomography subsystem (XCT) providing XCT anatomical information as structural a priori data to the integrated tri-modality system, while the diffuse optical tomography subsystem (DOT) provides optical background heterogeneity information from DOT measurements to the integrated tri-modality system as functional a priori data. A method includes using FT, DOT, and XCT in an integrated fashion wherein DOT data is acquired to recover the optical property of the whole medium to accurately describe photon propagation in tissue, where structural limitations are derived from XCT, and accurate fluorescence concentration and lifetime parameters are recovered to form an accurate image.

### RELATED APPLICATIONS

The present application is related to U.S. Provisional Patent Application Ser. No. 61/430,036, filed on Jan. 5, 2011, which is incorporated herein by reference and to which priority is claimed pursuant to 35 USC 119.

### GOVERNMENT RIGHTS

This invention was made with government support under EB007873 awarded by The National Institutes of Health. The government has certain rights in the invention.

### BACKGROUND

1. Field of the Technology

The disclosure relates to the field of apparatus and methods used for imaging biological specimens and in general small animal imaging, preclinical imaging systems, x-ray imaging, molecular imaging and optical imaging.

2. Description of the Prior Art

Fluorescence imaging has become an essential in vivo small animal imaging tool in recent years. Both commercial availability and relative ease of use has enabled widespread adoption of this technique. Most commercial fluorescence imaging systems utilize a planar imaging approach. However, the location, size and concentration of fluorescence inclusions buried deep in tissue cannot be obtained accurately from a two-dimensional projection image. This is due to the highly diffusive nature of light propagation in tissue. Fluorescence tomography (FT), on the other hand, is capable of generating a cross-sectional fluorophore distribution and lifetime maps.

In the last decade, researchers from various institutions have developed small animal fluorescence tomography systems for many applications. Despite rapid and promising development, the quantitative accuracy of FT technique is limited due to the ill-posed nature of the FT inverse problem. In order to improve the quantitative accuracy of FT techniques, several attempts have been made to combine FT with other anatomic imaging modalities such as magnetic resonance imaging (MRI) and x-ray computed tomography (XCT). In fact, the idea of combining a functional/molecular imaging modality with a high spatial resolution anatomical imaging modality has been applied previously to optical and nuclear imaging. Examples of this approach can be found in combined positron emission tomography (PET) and XCT systems and combined single photon computed tomography (SPECT) and MRI.

The advantage of such multi-modality approach is two-fold. First, functional information provided by nuclear imaging is perfectly coregistered with high resolution anatomical images and thus improves visualization of nuclear activity. Second, the structural a priori information can improve quantitative accuracy of the nuclear imaging by guiding and constraining the reconstruction algorithm.

Another example of multi-modality approach is combining DOT with MRI and X-ray mammography. Like PET, DOT is a low-resolution functional imaging modality, but uses near-infrared light to investigate the optical absorption and scattering properties of tissue. Up to date, extensive effort has been made to integrate DOT with other imaging modalities to obtain accurate optical property maps of the tissue under investigation. This approach again aims for obtaining more accurate functional and molecular information with the guidance of the high-resolution anatomical priors. Similarly, several studies have demonstrated importance of using structural information in fluorescence tomography. Simulation studies have shown that recovered fluorescence parameters can be improved when structural a priori information is used. There are two experimental approaches for obtaining structural a priori information along with FT. The first approach is to acquire FT and anatomical images in separate settings. The anatomical information is then coregistered and used to improve FT reconstruction. We have previously demonstrated that MR structural a priori information drastically improves the quantitative accuracy with a bench-top FT system. Other researchers have also developed a PMT based FT system and obtained the structural information using a XCT system. The second approach is to build integrated systems that can acquire both FT and anatomical images in the same setting. The key advantage of this approach is guaranteed accurate coregistration of the optical and anatomical images. For instance, the prior art has shown a hybrid FT/MRI system. Others have developed an integrated XCT-FT bench-top system. They demonstrated enhanced visualization of the fluorescence activity due to co-localization of FT and XCT images even without utilizing anatomical priors. Besides, prior art researchers have also presented that the fluorophore localization improves with XCT anatomical information using an integrated FT and XCT system.

Besides utilization of structural a priori information, FT quantitative accuracy can be improved further if the tissue optical heterogeneity is taken into account. The most common technique is to treat the tissue as a homogeneous medium after normalizing fluorescence measurements with respect to intrinsic excitation measurements. A more elaborate method is to obtain and use background optical property map to accurately model photon migration in both excitation and excitation wavelengths. In FT, excitation light propagation is modeled from the boundary to the fluorophore located inside the tissue first. Afterwards, emission light propagation is modeled from the fluorophore to the detectors located at the boundary of the tissue. Hence, optical properties at both excitation and emission wavelengths that are obtained by DOT can be used as functional a priori information prior to the reconstruction of FT parameters.

It has already been demonstrated that the fluorophore concentration is recovered more accurately when the background optical property map is provided. What is needed though is some kind of method and apparatus for improving the performance of FT by utilizing both structural and functional a priori information.

### BRIEF SUMMARY

In this disclosure, a first-of-its-kind fully integrated tri-modality system is described that combines fluorescence, diffuse optical and x-ray tomography (FT/DOT/XCT) into one instrument. This system performs quantitative fluorescence tomography using multimodality imaging. XCT anatomical information is used as structural a priori data while optical background heterogeneity information obtained by DOT measurements is used as functional a priori data. The performance of the hybrid system is evaluated using multi-modality phantoms. In particular, we show that a 2.4 mm diameter fluorescence inclusion located in a heterogeneous medium can be localized accurately with the functional a priori information, although the fluorophore concentration is recovered with 70% error. On the other hand, the fluorophore concentration can be accurately recovered within 8% error only when both DOT optical background functional and XCT structural a priori information are utilized to guide and constrain the FT reconstruction algorithm.

There has been no previous work utilizing fluorescence tomography (FT), diffuse optical tomography (DOT) and x-ray computed tomography (XCT) together in the method or in an integrated system to obtain quantitative fluorescence parameters. DOT alone has been used to guide FT, and XCT alone has been used to guide FT. However, the improvement is limited. Only when both DOT and XCT information are used are the reconstructed fluorescence parameters quantitatively accurate.

The inverse problem of fluorescence tomography is severely ill-posed, so the recovered fluorescence parameters are not at all quantitative. Previously, XCT has been used to provide anatomical a priori information to guide and constrain the FT reconstruction. Significant improvement over stand-alone FT has been shown using this approach. However, the improvement is limited due to the lack of the knowledge on the background optical property.

In our FT/DOT/XCT embodiment, DOT data is acquired to recover the optical property of the whole medium. With this step, the photon propagation in tissue is accurately described. Together with the structural guidance from XCT, accurate fluorescence parameters can be recovered.

The illustrated embodiment is directed to an apparatus and method for obtaining 3D fluorescence imaging of small animals in vivo. There are two main parts of the embodiment are instrumentation and software. The software uses the data collected by the instrument and reconstructs the fluorescence concentration image. For the hardware part, a gantry-based fluorescence tomography (FT) system provides cross sectional fluorescence concentration images. It uses a CCD camera and multiple lasers to acquire fluorescence images in transmission mode from several views. One unique property of the design is that it is compatible with x-ray computed tomography (X-ray CT). X-ray CT is also a gantry based imaging modality that provides anatomical tomographic images of the small animals that can be used as a priori information to improve the optical imaging quality.

The second property of the system is that it acquires diffuse optical tomography (DOT) data together with FT data. DOT provides the necessary background optical property map to improve FT image accuracy. Hence, this is by itself a multi-modality imaging modality system that combines FT and DOT techniques.

For the software part, we have developed a complete software system using Matlab to perform the reconstruction using an integrated FT/DOT/XCT system. One purpose of this system is to obtain quantitatively accurate fluorescence concentration images in vivo using a multi-modality approach. XCT offers anatomical information while DOT provides the necessary background optical property map to improve FT image accuracy.

The quantitative accuracy of this trimodal system is demonstrated in vivo in the illustrated embodiment. In particular, we show the fluorophore concentration of a 2 mm diameter fluorescence inclusion. located 8 mm deep in a nude mouse can be accurately recovered within 2% error when both DOT functional and XCT structural a priori information are utilized together to guide and constrain the FT reconstruction algorithm.

Thus, in summary the illustrated embodiments of the invention include an apparatus for providing an integrated tri-modality system including a fluorescence tomography subsystem (FT), a diffuse optical tomography subsystem (DOT), and an x-ray tomography subsystem (XCT), where each subsystem is combined in the integrated tri-modality system to perform quantitative fluorescence tomography with the fluorescence tomography subsystem (FT) using multimodality imaging with the x-ray tomography subsystem (XCT) providing XCT anatomical information as structural a priori data to the integrated tri-modality system, while the diffuse optical tomography subsystem (DOT) provides optical background heterogeneity information from DOT measurements to the integrated tri-modality system as functional a priori data.

The apparatus further includes a computer coupled to each of the subsystems and performing an FT reconstruction algorithm constrained by both DOT optical background functional and XCT structural a priori information.

In one embodiment the integrated tri-modality system includes a gantry and where the x-ray tomography subsystem (XCT), the fluorescence tomography subsystem (FT), and the diffuse optical tomography subsystem (DOT) are each mounted within or on the gantry.

The fluorescence tomography subsystem (FT) measures a fluorophore of a sample and includes an absorption and fluorescence laser operating at a corresponding wavelength based on the fluorophore to be measured, an optic switch to allow sequential activation of each laser, a plurality of optical outputs provided at the optic switch and collimators to allow illumination of the sample with a collimated beam at the corresponding wavelengths from a plurality of angles, a camera to capture an image of the sample, a controllable filter wheel coupled to the camera, and a controller or computer coupled to the camera and filter wheel.

The x-ray tomography subsystem (XCT) includes an x-ray source and x-ray detector, which are rotatable by the gantry along with the fluorescence tomography subsystem (FT).

The apparatus further includes a software controlled controller or computer communicated to the fluorescence tomography subsystem (FT), the diffuse optical tomography subsystem (DOT), and the x-ray tomography subsystem (XCT) to control each to perform automatic data acquisition.

The illustrated embodiments are further understood as including within their scope a method comprising the steps of performing fluorescence tomography (FT) of a biological specimen in an integrated (FT/XCT/DOT) system, performing diffuse optical tomography (DOT) of the biological specimen to provide a measurement of background optical property in the integrated (FT/XCT/DOT) system, performing x-ray computed tomography (XCT) of the biological specimen in the integrated (FT/XCT/DOT) system to provide anatomical a priori information, and reconstructing quantitative fluorescence parameters as constrained by both the diffuse optical tomography (DOT) of the specimen and the x-ray computed tomography (XCT) of the specimen.

The step of reconstructing quantitative fluorescence parameters as constrained by both the diffuse optical tomography (DOT) of the specimen includes modeling excitation and emission fluorescence light propagation in the biological specimen in a computer using a coupled diffusion equation:

$$\begin{matrix}∇ \cdot [ {D}_{x} ( \vec{r} ) ∇ {Φ}_{x} ( \vec{r} , ω ) ] - [ {μ}_{ax} ( \vec{r} ) + {μ}_{af} ( \vec{r} ) + \frac{i ω}{{c}_{n}} ] {Φ}_{x} ( \vec{r} , ω ) = - {q}_{0} ( \vec{r} , ω ) & ( 1 ) \\ ∇ \cdot [ {D}_{m} ( \vec{r} ) ∇ {Φ}_{m} ( \vec{r} , ω ) ] - [ {μ}_{am} ( \vec{r} ) + \frac{i ω}{{c}_{n}} ] {Φ}_{m} ( \vec{r} , ω ) = - {Φ}_{x} ( \vec{r} , ω ) η {μ}_{of} ( \vec{r} ) \frac{1 - i ω τ ( \vec{r} )}{1 + {[ ω τ ( \vec{r} ) ]}^{2}} & ( 2 )\end{matrix}$$

where φₓ(r) and φₘ(r) (W·mm⁻²) are the photon density for the excitation and emission light, respectively, where the diffusion coefficient, D_{x,m}(r) (mm⁻¹), is defined by D_{x,m}=⅓(μ_{a x,m}+μ_{s′ x,m}), where reduced scattering and the absorption coefficients of the specimen are μ_{s′ x,m} (mm⁻¹) and μ_{a x,m} (mm⁻¹), respectively, where the absorption coefficient due to fluorophore, μ_{af}(r), is related to the concentration C of the fluorophore by μ_{af}=2.3εC, where ε is the extinction coefficient of the fluorophore with the unit of Molar⁻¹·mm⁻¹ and C is the concentration of the fluorophore, where total absorption coefficient at excitation wavelength (μ_{a x}) includes the contribution from the fluorescence absorption μ_{af}(r), where quantum yield, η, is defined as the ratio of the number of photons emitted to the number of photons absorbed by the fluorophore. The modulation angular frequency and the speed of light in the tissue are represented by ω and cₙ, respectively. In addition the fluorescence lifetime is represented with τ. In the illustrated embodiment, we only focus on the fluorescence absorption, but inclusion of fluorescence lifetime τ is included within the scope of the invention.

The step of reconstructing quantitative fluorescence parameters includes solving the coupled diffusion equation in a computer using finite element method (FEM) and where the inverse problem is solved by minimizing the difference between the measured and calculated data according to the following error function:

$${ɛ}^{2} ( {μ}_{a} ) = {\sum }_{i = 1}^{{N}_{s}} {\sum }_{j = 1}^{{N}_{d}} {( {φ}_{ij}^{m} - {P}_{ij} ( {μ}_{a} ) )}^{2}$$

$${ɛ}^{2} ( {μ}_{af} ) = {\sum }_{i = 1}^{{N}_{s}} {\sum }_{j = 1}^{{N}_{d}} {( {φ}_{ij}^{m} - {P}_{ij} ( {μ}_{af}^{′} ) )}^{2}$$

for DOT and FT measurements, respectively, where, i represents the number of sources and j represents the number of detectors. φ^{m}ᵢⱼ is the measurement. Pᵢⱼ(μₐ) and Pᵢⱼ(μ_{af}) are the flux at a measured point calculated by the forward solver from the spatial distribution of μ_{ax,m} and μ_{af}.

The method further includes the step of iteratively updating the unknown μₐ and μ_{af} with Levenberg-Marquardt method by

Xₘ₊₁=Xₘ+(J^{T}J+λI)⁻¹(J^{T}ε)

where εᵢⱼ=(φ^{m}ᵢⱼ−Pᵢⱼ) and X represents the unknown matrix of μ_{ax,m} and μ_{af}, where the dimension of X is N and it represents the number of nodes in the FEM mesh, where the Jacobian matrix J is calculated with adjoint method.

The step of reconstructing quantitative fluorescence parameters as constrained by both the diffuse optical tomography (DOT) of the specimen and the x-ray computed tomography (XCT) of the specimen includes reconstructing μ_{ax,m} from the DOT data to correct optical background heterogeneity, and reconstructing μ_{af} using φ_{x,m} and μ_{ax,m} that are obtained from the DOT by assuming a homogeneous μ_{af} distribution as an initial guess and then generating new values of μ_{af} by minimizing the difference between the forward solver solution and the measurements using structural a priori information.

The illustrated embodiments further are understood to include a method comprising the steps of using fluorescence tomography FT, diffuse optical tomography DOT, and x-ray computed tomography XCT in an integrated fashion wherein DOT data is acquired to recover the optical property of the whole medium to accurately describe photon propagation in tissue, where structural limitations are derived from XCT, and accurate fluorescence parameters are recovered to form an accurate image.

The step of using fluorescence tomography FT, diffuse optical tomography DOT, and x-ray computed tomography XCT in an integrated fashion further comprises performing 3D fluorescence imaging of small animals in vivo.

The method further includes reconstructing a fluorescence concentration image.

The method further includes using a gantry-based fluorescence tomography (FT) system to provide cross sectional fluorescence concentration images.

The step of using fluorescence tomography FT, diffuse optical tomography DOT, and x-ray computed tomography XCT in an integrated fashion includes using a CCD camera and multiple lasers to acquire fluorescence images in a transmission mode from several views in a manner compatible with x-ray computed tomography (X-ray CT) to provide anatomical tomographic images of the small animals used as a priori information to improve the optical imaging quality.

The step of using fluorescence tomography FT, diffuse optical tomography DOT, and x-ray computed tomography XCT in an integrated fashion comprises acquiring diffuse optical tomography (DOT) data together with FT data, where the DOT data provides a background optical property map to improve FT image accuracy.

The step of using fluorescence tomography FT, diffuse optical tomography DOT, and x-ray computed tomography XCT in an integrated fashion includes using lasers to illuminate an object from three sides, using a cooled CCD camera as an optical detector, and a computer controlled filter wheel to automatically change optical bandpass filters for FT or DOT measurements.

The method further includes performing preclinical fluorescence molecular imaging to acquire quantitatively accurate fluorescence parameters for cancer imaging, stem cell imaging, cell therapy monitoring or drug development.

The step of using fluorescence tomography FT, diffuse optical tomography DOT, and x-ray computed tomography XCT in an integrated fashion includes providing increased accuracy of a recovered FT parameter without contact and acquiring other modality data in the same setting with exact co-registration.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

The specification contains at least one drawing executed in color. Copies of this patent or patent application publication with color drawing(s) will be provided by the Office upon request and payment of the necessary fee.

FIG. 1 is a schematic drawing of the combined FT/CT system of the illustrated embodiment showing a configuration of the optical and x-ray components. The view is along the axial direction of the gantry system of FIG. 2a and P represents the phantom/patient position.

FIG. 2a is a schematic diagram of the multi-modality gantry-based system of the illustrated embodiment.

FIG. 2b is a diagram of the light delivery components of the system of FIG. 2a.

FIG. 3a is a trans-axial XCT image of the phantom. The ICG inclusion is held in the glass tube that is seen as a bright circle in the image.

FIG. 3b is a graph of the recovered ICG concentration with respect to true ICG concentration. The circles and triangles represent the recovered values with and without the structural a priori information, respectively. The least squares lines of best fit are the dashed ones. The recovered ICG concentration shows a linear response with respect to true ICG concentration both with and without the structural a priori information. However, the right values are recovered only when structural a priori information is available.

FIG. 4 shows reconstructed ICG concentration maps without (left column) and with (right column) structural a priori information from XCT for the first phantom study. The calibration bars all have units of nM.

FIG. 5 illustrates the results for the second phantom study. The first column is the XCT trans-axial images of the phantoms. The size and location of the inclusion are different for each case. The second the third columns are the reconstructed ICG concentration maps without and with the XCT structural a priori information, respectively. As seen in the images, the recovered ICG concentration value depends drastically on the size and location of the inclusion. However, the true value can be recovered for all four cases when XCT structural a priori information is used. The calibration bars all have the units of nM.

FIG. 6a is an XCT trans-axial image of the heterogeneous phantom.

FIG. 6b is a reconstructed absorption map at 785 nm using DOT measurements.

FIG. 6c is a reconstructed ICG concentration map without any a priori information.

FIG. 6d is a reconstructed absorption map at 785 nm using DOT measurements with functional a priori information alone.

FIG. 6e is a reconstructed absorption map at 785 nm using DOT measurements with both functional and structural a priori information.

FIG. 6f is a profile plot along the x-axis across the reconstructed fluorescence object for each case is superimposed.

FIG. 6g is a profile plot along the x-axis across the glass tube (indicated by dashed line in the XCT image).

FIGS. 7a and 7b are reconstructed ICG concentration maps without and with XCT structural a priori information respectively.

FIG. 7c is a profile plot along the x-axis across the reconstructed fluorescence object for each case is superimposed.

FIG. 8a is an XCT trans-axial image of the phantom with two ICG inclusions.

FIGS. 8b and 8c are reconstructed ICG concentration maps without and with XCT structural a priori information. Without XCT structural a priori information, the object closer to the surface dominates in the image. On the other hand, both ICG inclusions can be accurately recovered when XCT structural a priori information is used.

FIG. 9 is a flow diagram illustrating the steps of the methodology of the illustrated embodiment.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

We have built a first-of-its-kind gantry-based multi-modality system 10 that combines FT, DOT and XCT in an integrated platform. The XCT offers anatomical information while the DOT provides optical background heterogeneity to improve the FT images further. The performance of the system 10 was evaluated using multi-modality phantoms. We first assessed the linearity of the system response using fluorescence inclusions with various concentrations located in a homogeneous, tissue-mimicking phantom. Next, size and location dependence of the recovered fluorophore concentration was investigated. For both studies, the fluorophore concentration maps reconstructed with and without XCT structural a priori information were compared. Finally, we investigated the recovery of the fluorophore concentration of the inclusion in the presence of background heterogeneity. We demonstrated that fluorophore concentration could be accurately recovered only when both functional (DOT) and structural (XCT) co-registered a priori information is available.

Materials and Methods

Instrumentation

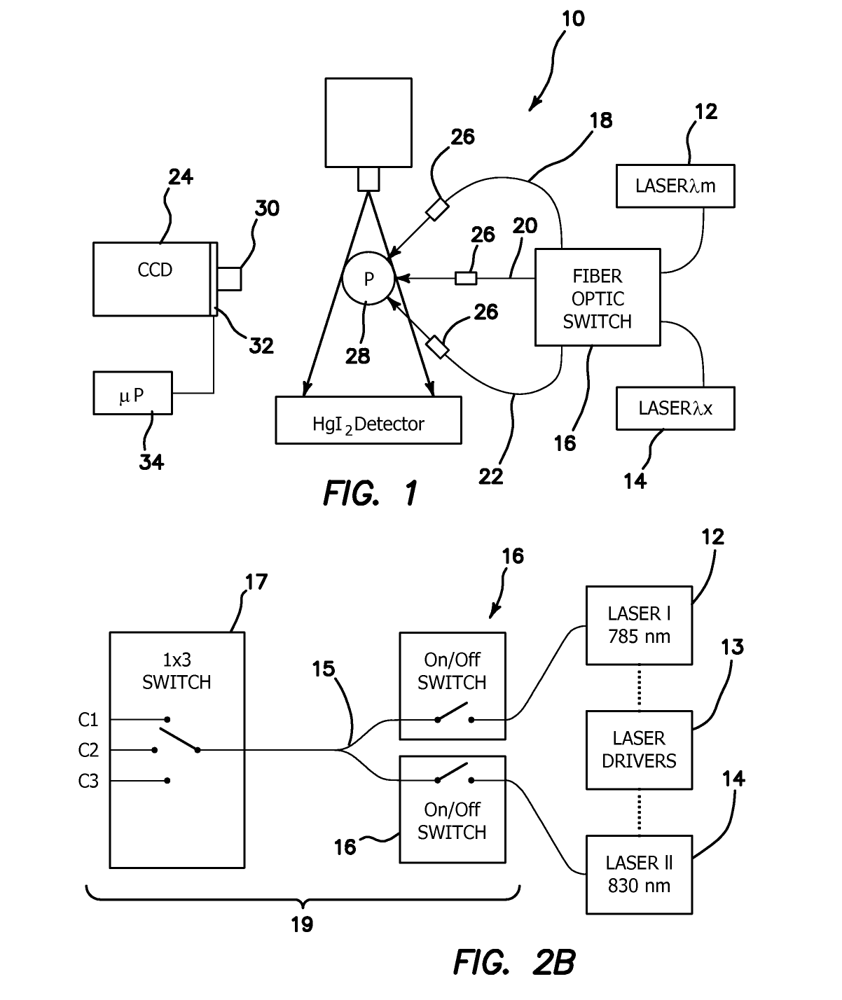

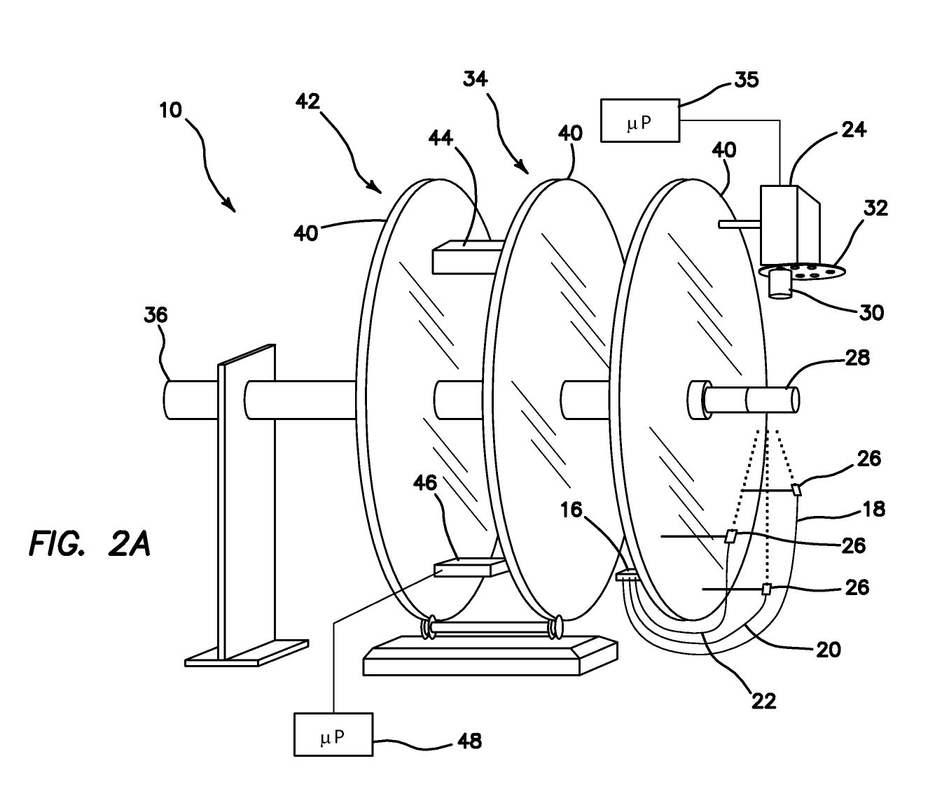

The XCT portion of the system 10 is a conventional x-ray system produced by DxRay, Inc (Northridge, Calif.). The rotatable XCT gantry 34 shown in FIG. 2a was expanded to install the optical imaging components for the tri-modality imaging system 10. A schematic of the laser delivery path of system 10 is shown in FIG. 1. Turn first to optical tomographic imaging. Both absorption and fluorescence measurements were carried out using the same optical instrumentation. Two different lasers 12 and 14 were used in the system, 785 nm (75 mW, Thorlabs) and 830 nm (150 mW, Intelite. Inc) respectively. The selection of the laser wavelengths was based on the optical property of the fluorophore used in the experiment, Indocyanine Green (ICG), which has the excitation and emission wavelength at 785 nm and 830 nm, respectively. It is to be expressly understood that that choice of laser frequencies can be varied using conventional principles depending on the intended application in hand to which system 10 is to be applied.

Conventional laser diode mounts and drivers 13 were installed on the gantry 34 as shown in FIG. 2b. The drivers 13 were operated at constant power mode to ensure the output stability during the experiments. An optical (on/off) switch 16 (Dicon Fiberoptics) was optically coupled with each laser 12 and 14 prior to combining their outputs. Both laser outputs were combined using a 50/50 fiber optic coupler 15 whose output was connected to a 1×3 fiber optic switch 17. The fiber optic switch 16 allowed sequential activation of each laser 12, 14. This configuration allowed us to illuminate the sample with a collimated beam at either 785 nm or 830 nm from three different angles (−45°, 0°, 45°) using optical output fibers 18, 20 and 22 in FIG. 1, each terminated with a collimator 26. A cooled CCD camera 24 (Perkin Elmer, Cold Blue) was also positioned on the gantry 34 across the three fiber optic collimators 26 and used to capture the images of the phantom 28. A sigma MACRO 50 mm F2.8 lens 30 was coupled to the CCD camera 24. A computer controlled filter wheel 32 (Tofra, Inc.) was installed between the CCD camera body 24 and the lens 30. Computer 35 was coupled to camera 24. One 830 nm band-pass filter (MK Photonics) and one long pass edge filter (Semrock, Inc) (not shown) were stacked between camera 24 and filter wheel 32 to eliminate excitation light at 785 nm. This filter stack combination minimizes the strong excitation leakage with maximum transmission rate at fluorophore emission wavelength.

FIG. 2a schematically illustrates the physical layout of the multi-modality gantry-based system 10. The XCT gantry 34 was expanded and optical imaging components were installed within the expanded gantry 34 as follows. A sample holder 36 was designed to translate the sample, which in the illustrated embodiment was a phantom, but typically would be the biological specimen of interest, between XCT and optical imaging subsystems. A sample holder 36 is used to move the phantom 28 by sliding a rod between isolating shields 40. The XCT subsystem 42 is includes x-ray source 44 and x-ray detector 46, which are rotatable by gantry 34 around the positioned sample or phantom 28. Also mounted to gantry 34 was the optical subsystem 48 including CCD camera 24, filter wheel 32, lens 30, fiber optic collimators 26, optical fibers 18, 20 and 22, and fiber optic switch 19, which is generally understood here to include the elements of FIG. 2b. On-off switches 16 were used to select the desired illumination wavelength. The 50/50 fiber optic coupler 15 was used to combine both laser outputs. A 1×3 fiber optic switch 17 allowed the sequential activation of any one the three source sites or collimators 26.

Labview Software (National Instruments, Austin, Tex.) was used to control each component and perform automatic data acquisition. A data acquisition card (NI-6550) was used to control the on/off switches 16. In addition to this, a motion controller (NI-7330) and a step motor driver (NI-7604) (not shown) were used to control the filter wheel 32. The gantry rotation and the 1×3 fiber optic switch 17 were controlled via serial ports connected to the computer 35, while the CCD camera 24 was connected to the computer 35 via a USB port. All electronic connections between the computer 35 and the components of the optical imaging system 10 were transferred through a wire harness belt (not shown). A graphical user interface was designed to rotate the gantry 34, activate the desired source position, and acquire the CCD images with or without excitation filters.

Turn now and consider the implementation of the X-ray computed tomography. The XCT subsystem 42 in the illustrated embodiment had its own dedicated computer 48 for data acquisition and analysis. The only common component between XCT and optical imaging subsystems 42 and 43 was the gantry control mechanism. Hence, the corresponding serial port connector was switched to the XCT computer 48 prior to the XCT acquisition. Again, the electrical connections between the XCT computer 48 and the components on the gantry 34 were transferred through a separate wire harness belt (not shown). The components coupled to or controlled by computer 48 were the x-ray tube 44, flat panel sensor 46 and step motors (not shown) that control the gantry rotation as well as the source-detector distance. The x-ray tube 44 was operated at 50 kVp and 0.5 mA. The flat panel sensor 46 had an active area of 12 cm×12 cm and pixel size of 50 μm (C7942GP, Hamamatsu Photonics). Planar images were acquired from 256 projections over 360° degree rotation in a set and shoot mode. Transaxial images were reconstructed using a Feldcamp cone beam filtered back projection algorithm. A standard box-car filter (not shown) was used.

Mathematical Framework for Optical Image Reconstruction

A coupled diffusion equation is used to model the excitation and emission fluorescence light propagation in tissue:

$$\begin{matrix}∇ \cdot [ {D}_{x} ( \vec{r} ) ∇ {Φ}_{x} ( \vec{r} , ω ) ] - [ {μ}_{ax} ( \vec{r} ) + {μ}_{af} ( \vec{r} ) + \frac{i ω}{{c}_{n }} ] {Φ}_{x} ( \vec{r} , ω ) = - {q}_{0} ( \vec{r} , ω ) & ( 1 ) \\ ∇ \cdot [ {D}_{m} ( \vec{r} ) ∇ {Φ}_{m} ( \vec{r} , ω ) ] - [ {μ}_{am} ( \vec{r} ) + \frac{i ω}{{c}_{n}} ] {Φ}_{m} ( \vec{r} , ω ) = - {Φ}_{x} ( \vec{r} , ω ) η {μ}_{af} ( \vec{r} ) \frac{1 - i ω τ ( \vec{r} )}{1 + {[ ω τ ( \vec{r} ) ]}^{2}} & ( 2 )\end{matrix}$$

where φₓ(r) and φₘ(r) (W·mm⁻²) are the photon density for the excitation and emission light, respectively. The diffusion coefficient, D_{x,m}(r) (mm⁻¹), is defined by D_{x,m}=⅓(μ_{a x,m}+μ_{s′ x,m}). The reduced scattering and the absorption coefficients of the medium are represented as μ_{s′ x,m} (mm⁻¹) and μ_{a x,m} (mm⁻¹), respectively. The absorption coefficients are expected to be different at excitation and emission wavelengths due to the diverse spectral dependence of the absorption of each individual tissue chromophore. Meanwhile, absorption coefficient due to fluorophore, μ_{af}(r), is directly related to the ICG concentration by the formula μ_{af}=2.3εC, where ε is the extinction coefficient of the fluorophore with the unit of Molar⁻¹·mm⁻¹ and C is the concentration of the fluorophore. The total absorption coefficient at excitation wavelength (μ_{a x}) includes the contribution from the fluorescence absorption μ_{af}(r). Quantum yield, η is the intrinsic property of the fluorophore and defined as the ratio of the number of photons emitted to the number of photons absorbed. The modulation angular frequency and the speed of light in the tissue are represented by ω and cₙ, respectively. The fluorescence lifetime is represented with τ. However, if frequency (ω≠0) or time-domain measurements are acquired, lifetime parameter can also be recovered accurately in addition to the concentration. Hence, the embodiments of the invention are not limited to fluorescence concentration parameters only.

The coupled diffusion equation is conventionally solved with the finite element method (FEM).

The inverse problem was solved by minimizing the difference between the measured and calculated data according to the following error function:

$${ɛ}^{2} ( {μ}_{a} ) = {\sum }_{i = 1}^{{N}_{s}} {\sum }_{j = 1}^{{N}_{d}} {( {φ}_{ij}^{m} - {P}_{ij} ( {μ}_{a} ) )}^{2}$$

$${ɛ}^{2} ( {μ}_{af} ) = {\sum }_{i = 1}^{{N}_{s}} {\sum }_{j = 1}^{{N}_{d}} {( {φ}_{ij}^{m} - {P}_{ij} ( {μ}_{af} ) )}^{2}$$

for DOT and FT measurements, respectively. Here, i represents the number of sources and j represents the number of detectors. φ^{m}ᵢⱼ is the measurement. Pᵢⱼ(μₐ) and Pᵢⱼ(μ_{af}) are the flux on the measured point calculated by the forward solver from the spatial distribution of μ_{ax,m} and μ_{af}. We iteratively updated the unknown μₐ and μ_{af} with Levenberg-Marquardt method by

Xₘ₊₁=Xₘ+(J^{T}J+λI)⁻¹(J^{T}ε)

where εᵢⱼ=(φ^{m}ᵢⱼ−Pᵢⱼ) and X represents the unknown matrix of μ_{ax,m} and μ_{af}. The dimension of X is N and it represents the number of nodes in the FEM mesh. The Jacobian matrix J is calculated with adjoint method.

The data analysis has been divided into the following steps:

For optical background heterogeneity correction, we first reconstruct μ_{ax,m} from the DOT data.

Following that, μ_{af} is reconstructed using φ_{x,m} and μ_{ax,m} that are obtained from the DOT. A homogeneous μ_{af} distribution is assumed as the initial guess in the reconstruction process. These values are found by minimizing the difference between the forward solver solution and the measurements.

When the structural a priori information is available, ‘Laplacian-type a priori’ developed by Yalavarthy et. al. is used to find the fluorophore concentration for the inclusion and the background. See P. K. Yalavarthy, B. W. Pogue, H. Dehghani, C. M. Carpenter, S. Jiang, and K. D. Paulsen, “Structural information within regularization matrices improves near infrared diffuse optical tomography,” Opt. Express 15(13), 8043-8058 (2007).

The L-matrix can be written as:

$${L}_{ij} = \{ \begin{matrix}0 & if i and j are not in the same region \\ 1 / {N}_{r} & if i and j are in the same region \\ 1 & if i = j\end{matrix}$$

where Nᵣ represents the number of nodes included in one region. Then the update equation can be expressed as:

Xₘ₊₁=Xₘ+(J^{T}J+λL^{T}L)⁻¹(J^{T}ε)

Dual mesh is used for reducing the computational time. A fine mesh (4225 nodes and 8192 elements) is used for the forward solver, while a coarse mesh (1089 nodes and 2048 elements) is used for the inverse problem for both DOT and FT.

Consider now a tissue simulating phantom study. Several phantom studies were carried out to evaluate the performance of the tri-modality system. Optical images were acquired from 8 views (45 degrees apart) for each source position. For DOT measurements, one-second CCD integration time was enough. However, FT measurements were obtained with sixty-second integration time for each source position. For each view, a set of 21 virtual detectors were placed uniformly on the corresponding boundary segment and mapped to the CCD as described in previous disclosure. The phantoms were prepared using agarose powder. Intralipid and Indian ink were added as optical scatterer and absorber, respectively. ICG (IC-Green, Akorn, Inc) was used as the fluorophore in this study. According to the literature, the ICG extinction coefficient at 785 nm and 830 nm is 130,000 Molar⁻¹·mm⁻¹ and 22,000 Molar⁻¹·mm⁻¹, respectively. Meanwhile, the quantum efficiency of ICG is 0.016. The clinical CT contrast agent Omnipaque (GE healthcare) was also added to different compartments of the phantom so that the structure of the phantom could be obtained from XCT scan and used as structural a priori information.

Three phantoms studies were undertaken. The first study was designed to evaluate the linearity of the system response. The second study was designed to assess the size and location dependence of the reconstruction results without and with the structural a priori information. The third case was designed to investigate the effect of both the functional and structural a priori information on the FT reconstruction. ICG inclusions were embedded in a homogeneous background for the first two studies, and in a heterogeneous optical background for the third study. A full set of data was acquired using homogeneous DOT and FT calibration phantoms at the end of each study. The optical properties of the DOT calibration phantom were set to μₐ=0.01 mm⁻¹ and μ_{s′}=0.8 mm⁻¹. The optical properties of the FT calibration phantom were similar. However, ICG (34 nM) was added for the latter case, which set the absorption due to this fluorophore to μ_{af}=0.001 mm⁻¹. A complete set of FT and DOT measurements were taken using the homogeneous FT and DOT calibration phantom. Then the experimental data (F_{measured}) is calibrated using the calibration phantom measurements (Fₕₒₘₒ_{_{—}}_{measured}) and

$${F}_{calibrated} = \frac{{F}_{measured}}{{F}_{homo \_ measured}} \times {F}_{homo \_ forw}$$

where the Fₕₒₘₒ_{_{—}}_{forw} is calculated by the forward solver. The calibrated data Fₕₑₜₑ_{_{—}}_{calibrated} is then fed into the inverse solver. This step takes into account for the data/model mismatch. More details are given in our previous disclosure.

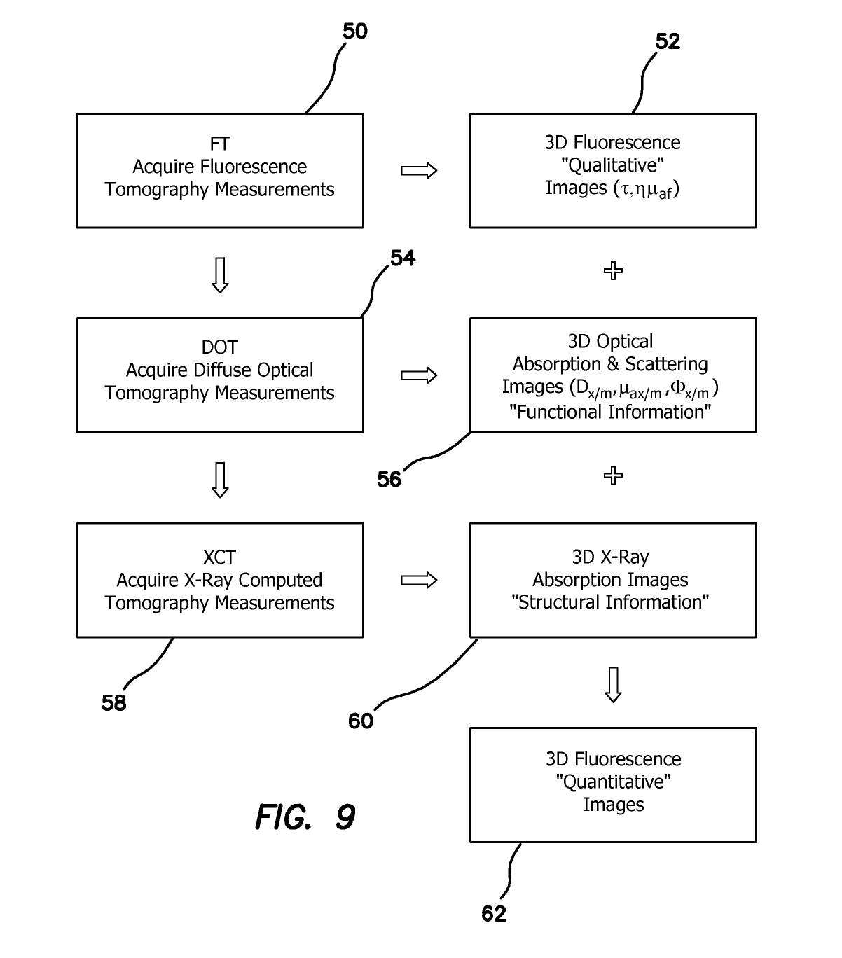

The methodology of the illustrated embodiment is shown in the flow diagram of FIG. 9. In step 50 the conventional fluorescence tomography (FT) measurements are obtained to obtain a qualitative three dimensional fluorescence concentration ημ_{af} and lifetime τ, images reconstructed at step 52. Diffuse optical tomography (DOT) is then performed at step 54 to obtain three dimensional absorption (μ_{ax,m}), scattering image (D_{x,m}) reconstruction as well as the photon density (φ_{x,m}) maps at step 56 to provide the functional information. X-ray computed tomography (XCT) is then performed at step 58 to obtain three dimensional x-ray image reconstruction at step 60 using to provide the structural information. The qualitative fluorescence information is combined with the DOT functional and XCT structural information at step 62 to obtain a reconstructed quantitative fluorescence image. Thus by using the DOT and XCT information, measured values for the functional and structural parameters, instead of estimates, can be inserted into the coupled fluorescence equations #1 and #2 and a quantitative fluorescence image reconstructed.

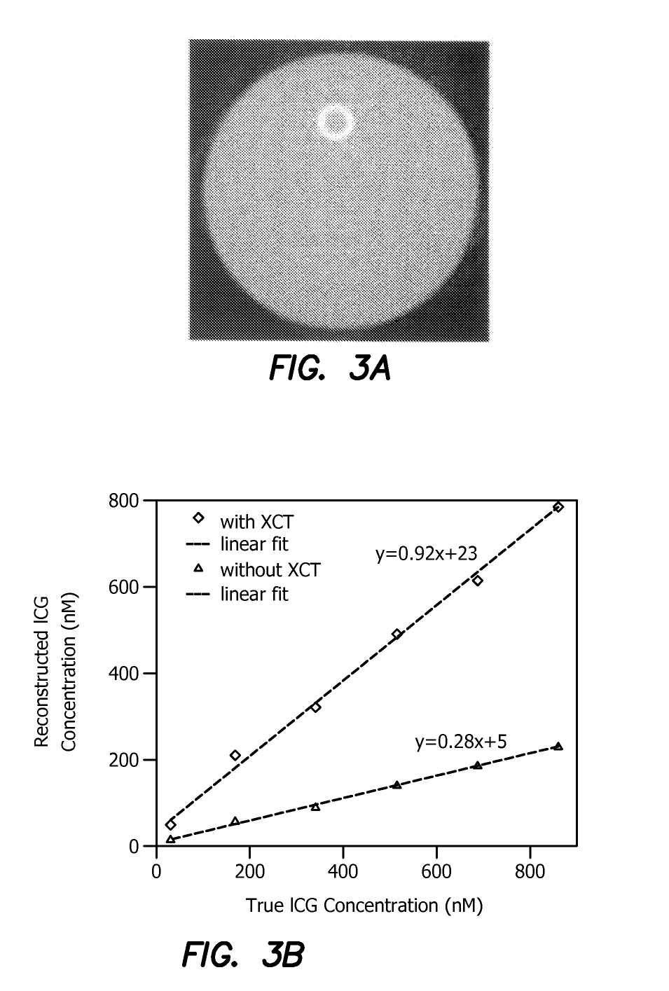

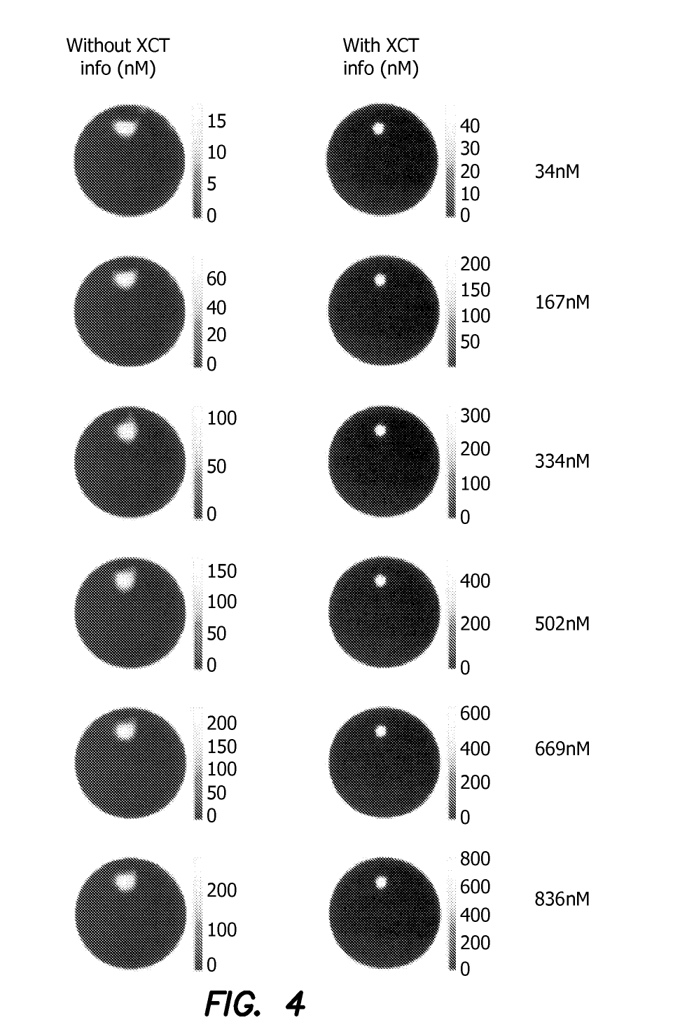

Turn now to the results of the phantom studies. Consider the phantom study I: response linearity. A 2.4 mm inner diameter glass tube filled with ICG and Intralipid is inserted in a 25 mm diameter phantom. The optical properties of the homogeneous background are set to μₐ=0.01 mm⁻¹ and μ_{s′}=0.8 mm⁻¹. The inclusion is 6 mm away from the center of the phantom. FIG. 3a shows the trans-axial XCT image of the phantom. The ICG concentration in the inclusion was varied in a broad range, from 34 nM to 830 nM, to evaluate the linearity of the system. The reconstructed ICG concentration maps without and with the structural a priori information are shown in the first and second columns in FIG. 4, respectively. The plot of the recovered ICG concentration with and without the structural a priori with respect to the true ICG concentration is shown in FIG. 3b.

As shown in FIG. 3b, the recovered fluorophore concentration is linear respect to the true concentration both with and without structural a priori information. The correlation coefficient is 0.998 for both fitted curves. However, the recovered ICG concentration is underestimated without the structural a priori information. On the other hand, the accuracy of the recovered ICG concentration is improved considerably for all the cases when the structural a priori information from XCT is used.

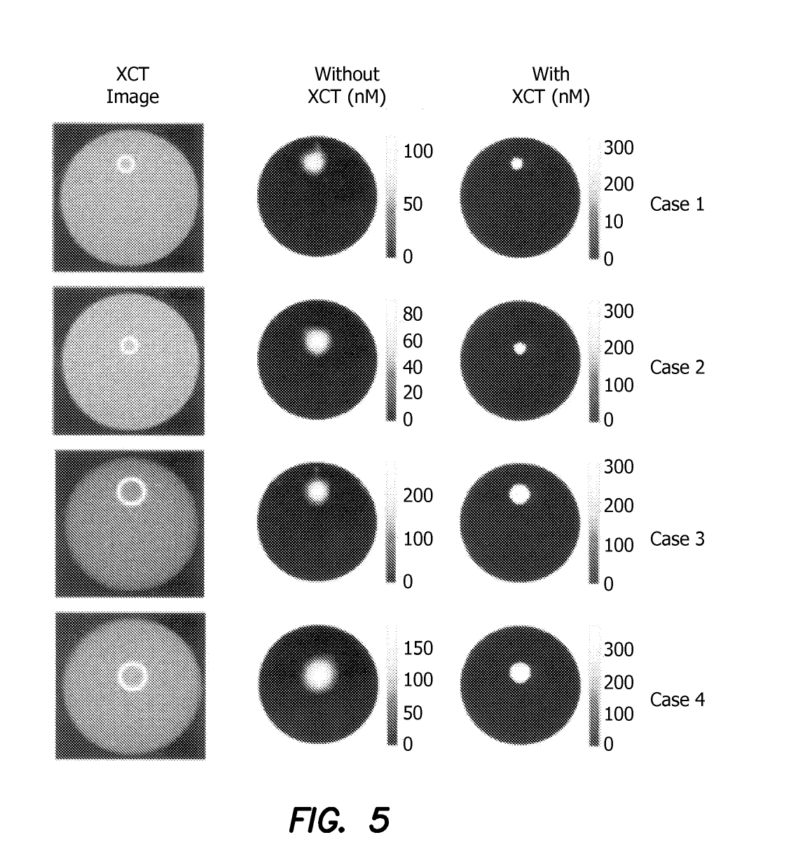

Consider the second phantom study and the results with respect to size and depth dependence. This study evaluates the effect of inclusion size and location on the recovered ICG concentration using four cases. The XCT images for the four cases are shown in the first column of FIG. 5. The first two cases both had a 2.4 mm ICG inclusion. The inclusions were positioned 7 mm and 2.5 mm off the center for the first and second case, respectively. Similarly, the third and fourth cases both have a 4.2 mm ICG inclusion. This time, inclusions were placed 6 mm and 2.5 mm away from the center. The background optical properties are kept the same as the first study. The ICG concentration in the inclusion was 334 nM for all four cases. Again, ICG concentration maps were reconstructed with and without the structural a priori information.

The recovered ICG concentration with and without the structural a priori information for all the four phantoms are listed in Table 1.

TABLE 1
The recovered ICG concentrations for phantom study 2.

| | | | | Recovered ICG | Recovered ICG | |
|---|---|---|---|---|---|---|
| | | | True ICG | concentration | concentration | |
| Case | Inclusion | Offset | concentration | (nM) without | (nM) with XCT | Error |
| # | size (mm) | (mm) | (nM) | XCT info | info | (%) |
| 1 | 2.4 | 7 | 334 | 96 | 326 | 2.4 |
| 2 | 2.4 | 2.5 | 334 | 70 | 328 | 1.8 |
| 3 | 4.2 | 6 | 334 | 232 | 302 | 9.6 |
| 4 | 4.2 | 2.5 | 334 | 157 | 357 | 6.9 |

For the small object (diameter 2.4 mm), the ICG concentration is recovered with 80% and 70% error when the inclusion is located 7 mm and 2.5 mm away from the center, respectively. For the 4.2 mm inclusion, on the other hand, the recovered ICG concentration has 50% error when it is located 2.5 mm off the center. The error was reduced to 30% when it was located 6 mm off the center. As expected, these results show that the recovered ICG concentration is more accurate when the inclusion is closer to the surface and its size larger. They also confirm that the recovered ICG concentration is highly dependent on the size and position of the inclusions, without structural a priori information. When structural a priori information from XCT is used to guide the reconstruction, however, the ICG concentration is recovered within 10% error for all cases, Table 1.

Consider the third phantom study III showing the results in the presence of heterogeneity. In this case, the ICG inclusion was embedded in a heterogeneous background. The background optical properties were μₐ=0.01 mm⁻¹ and μ_{s′}=0.8 mm⁻¹. The heterogeneous optical background was created by adding a 14 mm diameter object with the optical properties of μₐ=0.025 mm⁻¹ and μ_{s′}=0.8 mm⁻¹. This heterogeneous object was more absorptive than the background. The clinical available CT contrast agent Omnipaque was also added to the highly absorptive region to make it visible in the XCT image. In this study, a 2.4 mm diameter ICG inclusion was located 6 mm away from the center of the phantom. The absorption map of the phantom was reconstructed using diffuse optical tomography measurements and used as the functional a priori information. The ICG concentration map was reconstructed with three combinations of a priori information as follows:
i. The optical background heterogeneity was neglected, and ICG concentration map was reconstructed without functional and structural a priori information,
ii. The optical background was reconstructed with the DOT measurements and used as the functional a priori information. However, the structural a priori information was not used,
iii. Both the functional and structural a priori information were used during the reconstruction of ICG concentration map.

When the DOT functional a priori information was available, we first reconstructed μ_{x,m} from the DOT data. Then φₓ was calculated using μₓ and used in the second equation. Following that, μ_{af} was reconstructed using φₓ and μₘ that were obtained from the DOT. A homogeneous μ_{ax,m} or μ_{af} distribution was assumed as the initial guess in the reconstruction process. These values were found by minimizing the difference between the forward solver solution and the measurements.

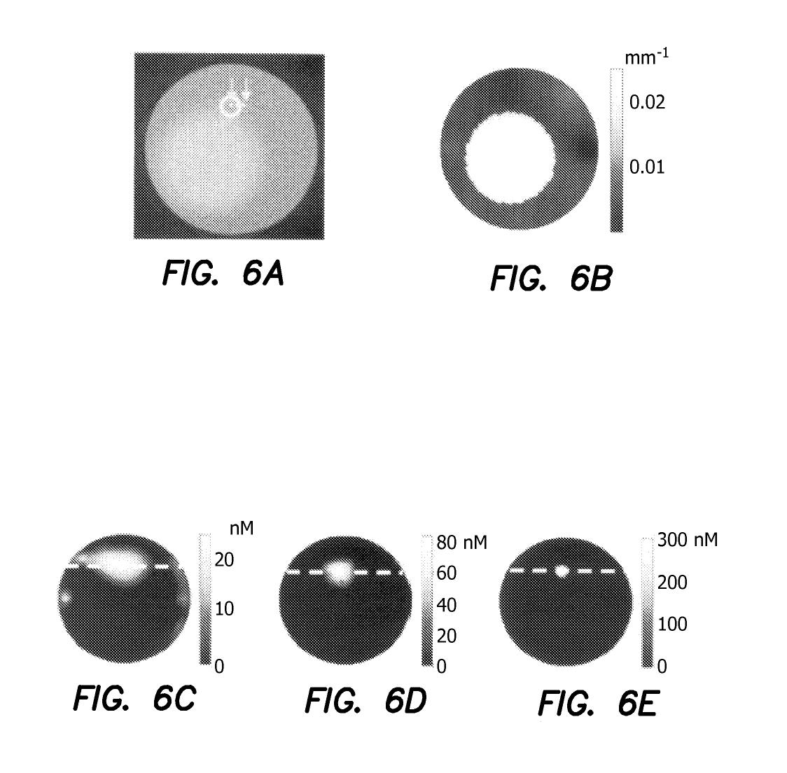

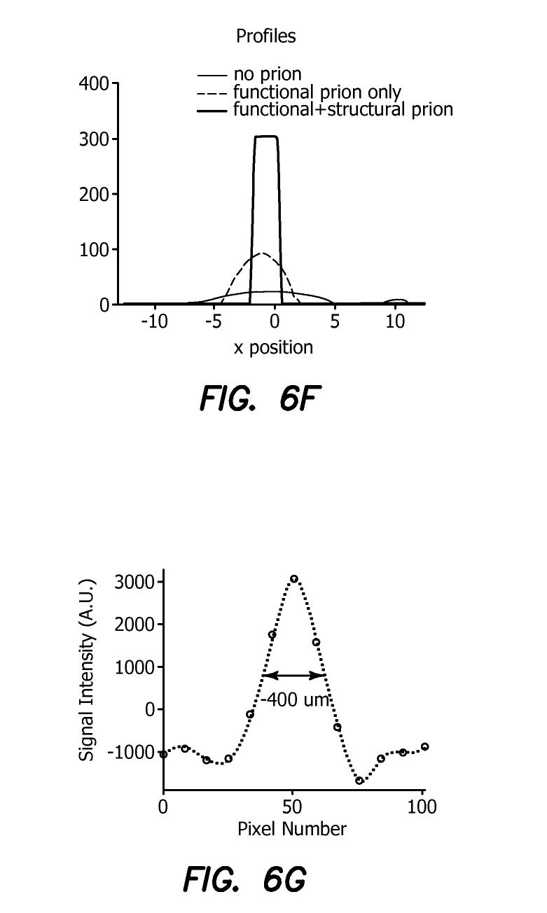

When the optical background heterogeneity is neglected, the shape of the reconstructed ICG object is distorted as shown in FIG. 6c. When functional a priori information from reconstructed DOT absorption map (FIG. 6b) is used, the ICG inclusion can be located accurately, FIG. 6d. However, the recovered ICG concentration shows 70% error. This is consistent with our second phantom study case 1. Please note that the mean recovered value of the absorptive region is 0.024 mm⁻¹, within 5% of its actual value. On the other hand, when both functional a priori information obtained from DOT and structural a priori information obtained from XCT are utilized, ICG concentration in the ICG inclusion can be recovered with only 8% error, FIG. 6e. The profile plot along the x-axis across the reconstructed fluorescence object for each case is superimposed and shown in FIG. 6f.

XCT is a high spatial resolution imaging modality. XCT spatial resolution is affected by many factors such as detector resolution (50 μm per pixel), x-ray source focal spot size (50 μm for our setting) and position of the object (magnification). To provide an estimate of the spatial resolution of the XCT system, we used the wall of the thin glass tube (300 μm) that was utilized as an inclusion in the phantom. For our XCT system, each pixel in the reconstructed cross-sectional XCT image corresponded to 150 μm, FIG. 6a. The full with half maximum (FWHM) of the curve (FIG. 6f) revealed that the resolution of the system was approximately 400 μm.

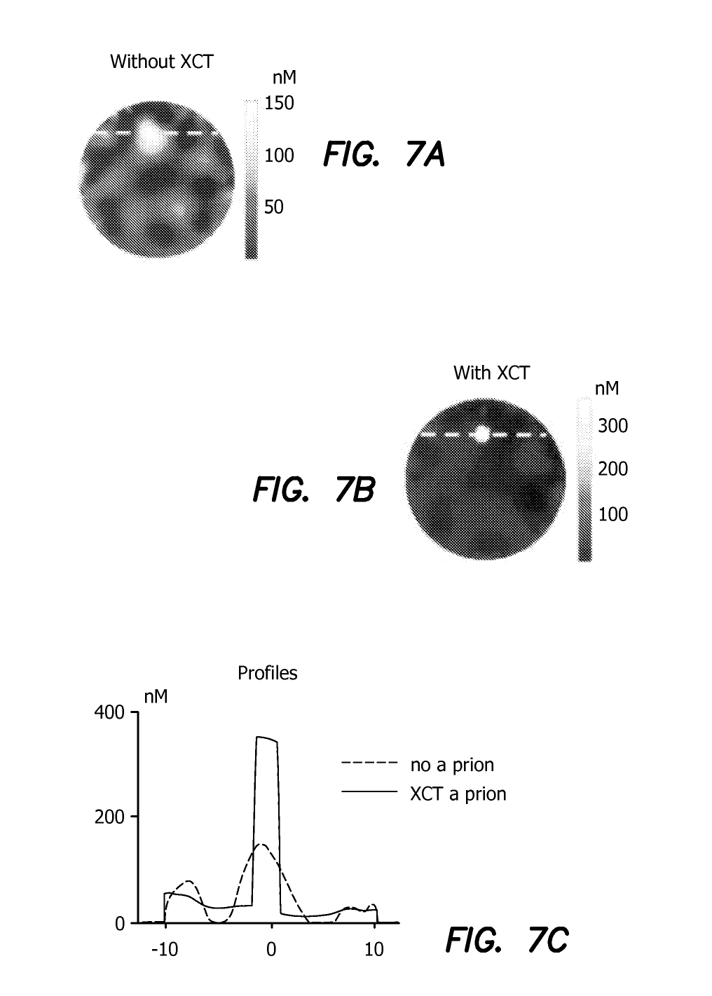

Turn to the fourth phantom study illustrating the presence of background fluorescence. One important aspect in fluorescence tomography studies is the contrast to background ratio (C/B), as in an in vivo setting there is likely to be residual fluorescence and/or autofluorescence from the tissues surrounding the fluorescent target. The reconstruction of fluorescence inclusion in the presence of background fluorescence is investigated using similar setting as the first phantom study. The optical properties of the 25 mm diameter homogeneous phantom are set to μₐ=0.01 mm⁻¹ and μ_{s′}=0.8 mm⁻¹. 34 nM ICG was also added to the background setting the absorption coefficient due to the fluorophore to be μ_{af}=0.001 mm⁻¹. Again, a 2.4 mm inner diameter glass tube filled with 334 nM ICG and Intralipid is inserted in the phantom. The XCT image of the phantom is the same as the one presented in FIG. 3a due to the use of the same phantom mold. The reconstructed ICG concentration maps without and with the structural a priori information are shown in the first and second columns in FIG. 7, respectively.

The inclusion can be localized in the presence of the background fluorescence even without XCT structural a priori information, as shown in FIG. 7a. Without any a priori info, the recovered ICG concentration for the target and the background is 84 nM (with 75% error) and 15 nM (with 55% error), respectively. On the other hand, when the XCT structural a prior information is used, the recovered ICG concentration for the target and the background is 341 nM (with 2% error) and 29 nM (with 14% error), FIG. 7b. The plot of intensity profiles along x axis across the reconstructed ICG inclusions is shown in FIG. 7c. The ability of this hybrid system to recover accurate ICG concentrations of multiple inclusions in the presence of the background is demonstrated in this study.

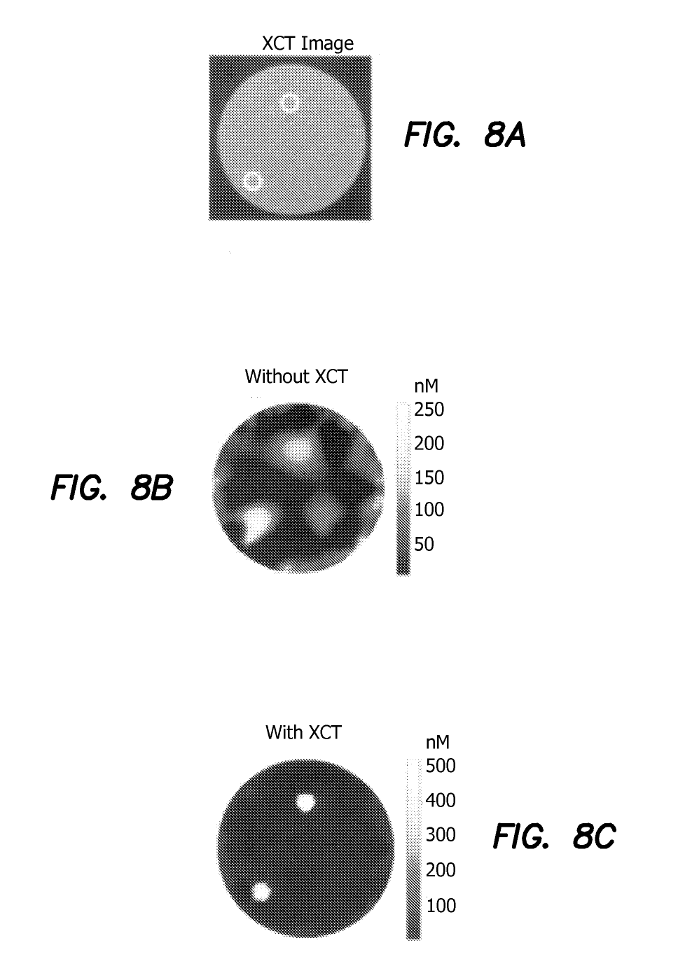

Consider the fifth phantom study V illustrating reconstruction of multiple fluorescence inclusions in the presence of background fluorescence. In the last phantom study, the reconstruction of multiple fluorescence inclusions in the presence of background fluorescence was investigated. The phantom had two 2.4 mm diameter ICG inclusions located 6.5 mm and 9 mm away from the center of the phantom as shown in XCT image (FIG. 8d), respectively. The background optical properties were μₐ=0.01 mm⁻¹, μ_{s′}=0.8 mm⁻¹ and μ_{af}=0.001 mm⁻¹ (34 nM ICG). Both inclusions have 500 nM ICG, making the contrast to background ratio 15. The reconstructed ICG concentration maps without and with the structural priori information are shown in the second and third columns in FIG. 8, respectively.

The two ICG inclusions are positioned at different depth; hence the recovered concentration values differ without XCT a priori information. The ICG concentration of the inclusions located 6.5 mm and 9 mm away from the center were recovered as 93 nM (with 81% error) and 167 nM (with 67% error), respectively. This is consistent with our second phantom study, in which we demonstrate that the recovered fluorophore concentration is highly dependent on the size and depth of the inclusion. In contrary, the error reduces down to 2% (508 nM) and 3% (513 nM) for the two inclusions when the XCT structural a priori information is utilized.

An ideal FT system should not only allow visualization of the fluorophore distribution in tissue but also provide quantitatively accurate concentration values in a heterogeneous medium. Quantitative accuracy is a pivotal factor for many practical applications of FT. For instance, MMPsense, which is an activatable fluorescence probe, accumulates three times higher in malignant tumors than benign ones in vivo. To be able to differentiate malignant and benign lesions, the reconstructed fluorophore concentration value should be independent of the size and location of the lesion. A stand-alone FT system, unfortunately, would not correctly differentiate a small malignant lesion buried deep in tissue and a large benign lesion located at subsurface lesion.

In order to address the need for quantitative FT imaging, we built a fully integrated FT/DOT/XCT system. There are two main objectives for developing this system. First, the XCT structural a priori information is used to guide and constrain the FT inverse problem, thus fluorophore concentration map can be recovered more accurately. Additionally, DOT provides an effective way of correcting the effect of optical background heterogeneity, thus improves the FT accuracy further.

Recently, others have particularly investigated the depth dependence of a subsurface FT technique with simulation and phantom studies. They concluded that the fluorescence object could be localized but recovered image sensitivity was nonlinear with depth. Our second phantom study also confirms that the recovered fluorescence concentration is highly dependent on not only the depth but also the size of the fluorescence inclusions. On the other hand, the fluorophore concentration in the inclusions can be recovered within 10% error using the XCT structural a priori information independent of their size and location.

Another factor that affects the FT reconstruction results is the background optical property distribution. Without proper modeling of emission and excitation light propagation between boundary of the medium and fluorophores, it is difficult to obtain accurate results. As an alternative, Born normalization method has been commonly used for heterogeneity correction, and applied to localize the fluorescence object robustly in vivo. For instance, other researchers have recovered a subcutaneously implanted fluorescence inclusion in nude mice with less than 30% error using slab geometry and matching fluid. However, the quantitative accuracy of Born normalization method has not been discussed especially for inclusions deeply embedded in the turbid medium. Furthermore, other researchers have particularly compared Born normalization and a heterogeneity correction method, which used reconstructed optical property for modeling purposes. The results showed that the later was more accurate. Our results also confirmed that significant improvement can be achieved when DOT functional a priori information is utilized during the FT reconstruction process. However, neither functional nor structural a priori by itself is enough to obtain accurate FT maps. In essence, the strength of the tri-modality system described here comes from its ability to offer both XCT structural and DOT functional a priori information that can be utilized to reconstruct quantitatively accurate fluorophore concentration images.

In our study, only absorption heterogeneity was considered and homogeneous scattering coefficient distribution was assumed. However, the scattering coefficient can also be very heterogeneous in reality. Time-dependent DOT measurements, which can be achieved using frequency- or a time-domain DOT system, are required in order to effectively separate the absorption and scattering coefficients. Nevertheless, the effect of the scattering error was evaluated using simulation studies. For example, when the scattering coefficient was chosen to be +/−25% of the actual value, the recovered absorption coefficient from DOT gives +/−28% for the absorptive object. In turn, when this absorption map is fed into the fluorescence reconstruction, the fluorescence concentration gives +/−30% error.

A perquisite for any such multimodality approach is that the region of interest should be detected by each individual modality. This may not be true all the time and even if it is, the boundary of the ROI detected by different modalities may vary. As a solution for the latter case, soft a priori approach is used to reduce the effect of erroneous a priori information on the reconstruction results. For the former case, XCT structural information can at least improve the optical property of the background medium obtained by DOT, which in turn will improve the FT results further. All the XCT images shown in this study is to provide phantom structure for FT reconstruction and demonstrate the importance of such multi-modality imaging system using proof-of-principle experiments. However, the limitation and optimized contrast and geometry for acquiring XCT image need to be addressed and investigated using contrast-detail analysis method in the future. Furthermore, various strategies such as utilization of XCT contrast agents or dual energy XCT technique can be potentially used to improve the vascular or soft tissue contrast. The most favorable case for such multimodality imaging system is to use dual modality contrast agent where the location of the agent can be seen from the structural imaging modality and the amount of the agent can be quantified by the fluorescence imaging. This is possible with the advances of dual contrast agent development, such as dual MRI-fluorescence, dual XCT-fluorescence contrast agent. Meanwhile, extensive effort is being spent to develop new x-ray detector technology to improve XCT soft tissue contrast further.

The illustrated embodiment of the invention is mainly intended for, but not limited to, preclinical fluorescence molecular imaging. The ability to acquire quantitatively accurate fluorescence parameters enable this method to be used for cancer imaging, stem cell imaging, cell therapy monitoring and drug development.

There is a large group of potential users of this technology. There are a number of traditional stand-alone fluorescence imaging systems on the market such as the makers of IVIS imaging systems, in vivo FT systems, and Fc systems. Mostly, these prior art stand-alone FT systems can only provide projection images and only one or two of them can provide cross-sectional images in tomographic mode. Our approach improves the quantitative accuracy of fluorescence tomography using additional DOT data. Furthermore, it can get superior images if it is coupled with another anatomic imaging modality such as X-ray CT. Prior art makers already have commercial modular (CT, PET, SPECT) animal imaging systems. One of these modules is an X-ray micro-CT system for small animal imaging, Hence, our FT-DOT system can be coupled to this module easily or sold as an additional module to the labs that are interested in optical imaging of small animals.

The advantages of the illustrated embodiment include: 1. Much better accuracy of the recovered FT parameter; 2. No required contact; and 3. Data acquired in the same setting, and perfect co-registration.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. An apparatus for providing an integrated tri-modality system comprising:
a fluorescence tomography subsystem (FT);
a diffuse optical tomography subsystem (DOT); and
an x-ray tomography subsystem (XCT),
where each subsystem is combined in the integrated tri-modality system to perform quantitative fluorescence tomography with the fluorescence tomography subsystem (FT) using multimodality imaging with the x-ray tomography subsystem (XCT) providing XCT anatomical information as structural a priori data to the integrated tri-modality system, while the diffuse optical tomography subsystem (DOT) provides optical background heterogeneity information from DOT measurements to the integrated tri-modality system as functional a priori data.

2. The apparatus of claim 1 further comprising a computer coupled to each of the subsystems and performing an FT reconstruction algorithm constrained by both DOT optical background functional and XCT structural a priori information.

3. The apparatus of claim 1 where the integrated tri-modality system comprises a gantry and where the x-ray tomography subsystem (XCT), the fluorescence tomography subsystem (FT), and the diffuse optical tomography subsystem (DOT) are each mounted within or on the gantry.

4. The apparatus of claim 1 where the fluorescence tomography subsystem (FT) measures a fluorophore of a sample and comprises an absorption and fluorescence laser operating at a corresponding wavelength based on the fluorophore to be measured, an optic switch to allow sequential activation of each laser, a plurality of optical outputs provided at the optic switch and collimators to allow illumination of the sample with a collimated beam at the corresponding wavelengths from a plurality of angles, a camera to capture an image of the sample, a controllable filter wheel coupled to the camera, and a controller or computer coupled to the camera and filter wheel.

5. The apparatus of claim 4 where the x-ray tomography subsystem (XCT), comprises an x-ray source and x-ray detector, which are rotatable by the gantry along with the fluorescence tomography subsystem (FT).

6. The apparatus of claim 1 further comprising a software controlled controller or computer communicated to the fluorescence tomography subsystem (FT), the diffuse optical tomography subsystem (DOT), and the x-ray tomography subsystem (XCT) to control each to perform automatic data acquisition.

7. A method comprising:
performing fluorescence tomography (FT) of a biological specimen in an integrated (FT/XCT/DOT) system;
performing diffuse optical tomography (DOT) of the biological specimen to provide a measurement of background optical property in the integrated (FT/XCT/DOT) system; and
performing x-ray computed tomography (XCT) of the biological specimen in the integrated (FT/XCT/DOT) system to provide anatomical a priori information; and
reconstructing quantitative fluorescence parameters as constrained by both the diffuse optical tomography (DOT) of the specimen and the x-ray computed tomography (XCT) of the specimen.

8. The method of claim 7 where reconstructing quantitative fluorescence parameters as constrained by both the diffuse optical tomography (DOT) of the specimen comprises modeling excitation and emission fluorescence light propagation in the biological specimen in a computer using a coupled diffusion equation:
$$\begin{matrix}∇ \cdot [ {D}_{x} ( \vec{r} ) ∇ {Φ}_{x} ( \vec{r} , ω ) ] - [ {μ}_{ax} ( \vec{r} ) + {μ}_{af} ( \vec{r} ) + \frac{i ω}{{c}_{n}} ] {Φ}_{x} ( \vec{r} , ω ) = - {q}_{0} ( \vec{r} , ω ) & ( 1 ) \\ ∇ \cdot [ {D}_{m} ( \vec{r} ) ∇ {Φ}_{m} ( \vec{r} , ω ) ] - [ {μ}_{am} ( \vec{r} ) + \frac{i ω}{{c}_{n}} ] {Φ}_{m} ( \vec{r} , ω ) = - {Φ}_{x} ( \vec{r} , ω ) η {μ}_{af} ( r ) \frac{1 - i ω τ ( \vec{r} )}{1 + {[ ω τ ( \vec{r} ) ]}^{2}} & ( 2 )\end{matrix}$$
where φₓ(r) and φₘ(r) (W·mm⁻²) are the photon density for the excitation and emission light, respectively, where the diffusion coefficient, D_{x,m}(r) (mm⁻¹), is defined by D_{x,m}=⅓(μ_{a x,m}+μ_{s′ x,m}), where reduced scattering and the absorption coefficients of the specimen are represented as μ_{s′ x,m} (mm⁻¹) and μ_{a x,m} (mm⁻¹), respectively, where the absorption coefficient due to fluorophore, μ_{af}(r), is related to the concentration C of the fluorophore by μ_{af}=2.3εC, where ε is the extinction coefficient of the fluorophore with the unit of Molar⁻¹·mm⁻¹ and C is the concentration of the fluorophore, where total absorption coefficient at excitation wavelength (μ_{a x}) includes the contribution from the fluorescence absorption μ_{af}(r), where quantum yield, η, is defined as the ratio of the number of photons emitted to the number of photons absorbed by the fluorophore, where ω is the modulation angular frequency and cₙ the speed of light in the specimen where fluorescence lifetime is τ.

9. The method of claim 8 where reconstructing quantitative fluorescence parameters comprises solving the coupled diffusion equation in a computer using finite element method (FEM) and where the inverse problem is solved by minimizing the difference between the measured and calculated data according to the following error function:
$$\begin{matrix}{ɛ}^{2} ( {μ}_{a} ) = {\sum }_{i = 1}^{{N}_{s}} {\sum }_{j = 1}^{{N}_{d}} {( {φ}_{ij}^{m} - {P}_{ij} ( {μ}_{a} ) )}^{2} & ( 3 ) \\ {ɛ}^{2} ( {μ}_{af} ) = {\sum }_{i = 1}^{{N}_{s}} {\sum }_{j = 1}^{{N}_{d}} {( {φ}_{ij}^{m} - {P}_{ij} ( {μ}_{af} ) )}^{2} & ( 4 )\end{matrix}$$
for DOT and FT measurements, respectively, where, i represents the number of sources and j represents the number of detectors. φ^{m}ᵢⱼ is the measurement. Pᵢⱼ(μₐ) and Pᵢⱼ(μ_{af}) are the flux at a measured point calculated by the forward solver from the spatial distribution of μ_{ax,m} and μ_{af}.

10. The method of claim 9 further comprising iteratively updating the unknown μₐ and μ_{af} with Levenberg-Marquardt method by
Xₘ₊₁=Xₘ+(J^{T}J+λI)⁻¹(J^{T}ε) (5)
where εᵢⱼ=(φ^{m}ᵢⱼ−Pᵢⱼ) and X represents the unknown matrix of μ_{ax,m} and μ_{af}, where the dimension of X is N and it represents the number of nodes in the FEM mesh, where the Jacobian matrix J is calculated with adjoint method.

11. The method of claim 7 where reconstructing quantitative fluorescence parameters as constrained by both the diffuse optical tomography (DOT) of the specimen and the x-ray computed tomography (XCT) of the specimen comprises:
reconstructing μ_{ax,m} from the DOT data to correct optical background heterogeneity; and
reconstructing μ_{af} using φ_{x,m} and μ_{ax,m} that are obtained from the DOT by assuming a homogeneous μ_{af} distribution as an initial guess and then generating new values of μ_{af} by minimizing the difference between the forward solver solution and the measurements using structural a priori information.

12. A method comprising using fluorescence tomography FT, diffuse optical tomography DOT, and x-ray computed tomography XCT in an integrated fashion wherein DOT data is acquired to recover the optical property of the whole medium to accurately describe photon propagation in tissue, where structural limitations are derived from XCT, and accurate fluorescence parameters are recovered to form an accurate image.

13. The method of claim 12 where using fluorescence tomography FT, diffuse optical tomography DOT, and x-ray computed tomography XCT in an integrated fashion further comprises performing 3D fluorescence imaging of small animals in vivo.

14. The method of claim 12 further comprising reconstructing a fluorescence concentration or fluorescence lifetime image.

15. The method of claim 12 further comprising using a gantry-based fluorescence tomography (FT) system to provide cross sectional fluorescence concentration and lifetime images.

16. The method of claim 12 where using fluorescence tomography FT, diffuse optical tomography DOT, and x-ray computed tomography XCT in an integrated fashion comprises using a CCD camera and multiple lasers to acquire fluorescence images in a transmission mode from several views in a manner compatible with x-ray computed tomography (X-ray CT) to provide anatomical tomographic images of the small animals used as a priori information to improve the optical imaging quality.

17. The method of claim 12 where using fluorescence tomography FT, diffuse optical tomography DOT, and x-ray computed tomography XCT in an integrated fashion comprises acquiring diffuse optical tomography (DOT) data together with FT data, where the DOT data provides a background optical property map to improve FT image accuracy.

18. The method of claim 12 where using fluorescence tomography FT, diffuse optical tomography DOT, and x-ray computed tomography XCT in an integrated fashion comprises using lasers to illuminate an object from three sides, using a cooled CCD camera as an optical detector, and a computer controlled filter wheel to automatically change optical bandpass filters for FT or DOT measurements.

19. The method of claim 12 further comprising performing preclinical fluorescence molecular imaging to acquire quantitatively accurate fluorescence parameters for cancer imaging, stem cell imaging, cell therapy monitoring or drug development.

20. The method of claim 12 where using fluorescence tomography FT, diffuse optical tomography DOT, and x-ray computed tomography XCT in an integrated fashion comprises providing increased accuracy of a recovered FT parameter without contact and acquiring other modality data in the same setting with exact co-registration.
